(19) United States
(12) Patent Application Publication
(10) Pub. No.: US 2024/0000592 A1
(43) Pub. Date: Jan. 4, 2024

(54) DEVICE AND METHOD FOR FENESTRATION ALIGNMENT

(71) Applicants: Massachusetts Institute of Technology, Cambridge, MA (US); Beth Israel Deaconess Medical Center, Inc., Boston, MA (US)

(72) Inventors: Tom McGlennon Dillon, Cambridge, MA (US); Patric Liang, Brookline, MA (US); Ellen Roche, Cambridge, MA (US); Marc Schermerhorn, Needham, MA (US)

(21) Appl. No.: 18/254,045

(22) PCT Filed: Nov. 23, 2021
(86) PCT No.: PCT/US2021/060591

§ 371 Date: May 23, 2023

Related U.S. Application Data
(60) Provisional application No. 63/117,246, filed on Nov. 23, 2020.
(60) Provisional application No. 63/153,803, filed on Feb. 25, 2021.
(60) Provisional application No. 63/195,464, filed on Jun. 1, 2021.

(51) Int. Cl. A61F 2/954; A61F 2/48; A61B 6/00; A61B 5/055

(52) U.S. Cl. CPC A61F 2/954; A61F 2/482; A61B 6/504; A61B 5/055; A61F 2002/061

(57) ABSTRACT

Patient-specific implants with fenestration(s) positioned at locations specific to patient's anatomy at the site of implantation are made using automated methods for making the patient-specific implants through fenestration alignment. Devices for making the patient-specific implants are also described. Typically, the devices operate on input of the fenestration alignment derived from the automated method. The devices and methods may form patient-specific linear (isodiametric) as well as patient-specific tapered (heterodiametric) implants with fenestrations.

### CROSS-REFERENCE TO RELATED APPLICATIONS

This application claims the benefit of and priority to U.S. Provisional Application No. 63/117,246 filed Nov. 23, 2020, U.S. Provisional Application No. 63/153,803 filed Feb. 25, 2021, and U.S. Provisional Application No. 63/195,464 filed Jun. 1, 2021, which are hereby incorporated by reference in their entirety.

### STATEMENT REGARDING FEDERALLY SPONSORED RESEARCH

This invention was not made with Government support.

### FIELD OF THE INVENTION

The invention is generally directed to devices and methods for making and using fenestrated aortic endograft implants.

### BACKGROUND OF THE INVENTION

The aorta is the largest artery in the body and distributes oxygen-rich blood away from the left ventricle to the body. The aorta is divided into the thoracic and abdominal aorta, in reference to the portion of the aorta in the chest and in the abdomen, respectively. An abdominal aortic aneurysm (AAA) results from dilation and degeneration of the aortic wall and is limited to the abdominal portion of the aorta, whereas a thoracoabdominal aortic aneurysm (TAAA) involves both the thoracic and abdominal segments of the aorta. Risk factors for developing an aortic aneurysm include smoking, high blood pressure, atherosclerosis, genetic diseases that weaken connective tissue in the body (e.g., Marfan's syndrome and Ehler Danos syndrome), and injury to the aortic wall such as aortic dissections.

A minimally invasive option to repair AAAs involve the use of an endovascular stent graft—a compliant tubular material reinforced with a metal stent mesh that can be inserted percutaneously through the femoral vessels. In the case of an infrarenal AAA (an aneurysm below the renal arteries), it is possible to repair the aneurysm using standard commercially available infrarenal stent grafts because there is adequate aortic length below the renal arteries to provide proximal seal for the stent graft. However, in the case of a juxtarenal or thoracoabdominal aneurysm (an aneurysm that extends across the visceral arteries that supply blood to major abdominal organs including the liver, stomach, kidneys, and intestines), standard infrarenal devices are contraindicated given the lack of aortic seal above these arteries to exclude the aneurysm and the need to perfuse the branches to the various arteries in the abdomen adjacent to the aneurysm if a graft is extended more proximally in the aorta.

Endovascular repair of these types of aneurysms therefore would require a fenestrated, patient-specific, stent graft to maintain perfusion to the visceral vessels. It is critical to ensure that these fenestrations, or holes in the graft, are unobstructed (i.e., their patency is maintained), to allow for continuous blood flow to the visceral vessels to prevent acute ischemia and fatal organ failure. Some medical device companies have started to manufacture patient-specific fenestrated grafts for customized treatment of juxtarenal AAA and TAAA. However, these grafts are only available to select users across the United States. Furthermore, the lead time can be on the order of a few weeks, so this technology is not appropriate for a patient who presents with an urgent condition that requires immediate surgical intervention.

Physician modified endografts can instead be performed by a surgeon by manually creating fenestrations onto a commercial tube endograft based on patient aortic anatomy. However, the current process of determining fenestration placement is cumbersome and prone to human-error. Therefore, there is a need for rapid reconstruction and fenestration alignment of a patient-specific fenestrated graft using preoperative computed tomography imaging.

There remains a need for a rapid reconstruction and alignment of a patient-specific fenestrated graft that allows blood flow into arteries branching from the aorta. It is an object of the present invention to provide methods of making the patient-specific fenestrated implants.

It is the object of the present invention to provide a computational method for assessing optimal fit of visceral fenestrations onto off the shelf, commercially available vascular endografts to generate customized patient-specific modifications.

It is another object of the present invention to provide methods for patient-specific fenestration alignment on the implants.

### SUMMARY OF THE INVENTION

Fenestration(s) positioned at locations specific to a patient's aortic anatomy and within endograft structural constraints have been developed. Automated methods and devices for making the patient-specific implants through fenestration alignment have also been developed. The method presents the first automated method for determining patient-specific fenestration alignment on fenestrated implants.

The patient-specific implants with fenestration(s) are made by an automated method for fenestration alignment on a medical implant. The automated method typically includes obtaining fenestration location and radius from patient's aortic anatomy, aligning the fenestration location and radius on an off-the-shelf medical implant such that the aligning does not collide with a structural component of the implant, and marking the fenestration location and marking the fenestration location on the medical implant.

Typically, obtaining the fenestration location and radius from the patient's anatomy requires obtaining Proximal Graft Distance (PGD) and Arclength (AL) for each fenestration. The patient's anatomy is typically determined with use of non-invasive imaging, namely computerized tomography angiography (CTA). The patient's anatomy is determined with a non-invasive imaging of computerized tomography (CT), magnetic resonance imaging (MRI), or radiography of the implantation site.

Typically, the aligning process is an automated process utilizing a program, such as a computer program or software, for aligning the fenestration location and radius on the medical implant. The aligning process includes digital operations such as positioning the fenestration location on a two-dimensional reconstruction of the implant, viewing, storing, modifying, printing, sharing, and three-dimensional reconstruction of the medical implant with fenestration location.

The methods may be used to produce fenestrated medical implants such as stents, implants, valves, vascular grafts, autologous grafts, allogeneic grafts, xenogeneic grafts, synthetic grafts, and hybrid stent grafts. The fenestrated medical implants typically do not have fenestrations over struts, wires, and stitches. This is because the method aligns the fenestrations such that they do not collide (touch or overlap) with the structural component of an implant, such as with any of struts, wires, and stitches.

The devices for marking a fenestration location on a medical implant typically include a rotating rod or mandrel for receiving the medical implant, a marker tool for marking the fenestration location, and one or more motors operably connected with the rotating rod or mandrel and/or the marker tool, to mark the fenestration location relative to the patient's vasculature as well as the struts or other reinforcing means in the graft, as needed, reinforcing the fenestration, and scanning to confirm strut locations with fenestration adjustments.

Typically, the one or more motors, when in operation, receive input of the fenestration alignment derived by the method. The marker tool is operably connected to the one or more motors for positioning the marker tool at the fenestration location. The one or more motors are typically operably connected with the marker tool via lead screws. The motors position the marker tool over the rotating rod. The medical implant may be positioned over the rotating rod, or over a. The device, when in operation, marks the implant at the fenestration locations derived by the method. The marker tool may be a marker or a cutting and/or cauterizing tool. The system may also include a device to reinforce the patient specific fenestration and a scanning device to confirm that there is no interference with struts in the graft after modification.

The devices and methods can be used to form patient-specific linear (isodiametric) implants with fenestrations as well as patient-specific tapered (heterodiametric) implants with fenestrations. An automated method for extracting the location of fenestrations from DICOM images may also be utilized in the pre-processing phase, to be made compatible with the fenestration alignment method (FAM). In post-processing, an automated method for aligning and rendering the 3D fenestrated graft that conforms inside 3D segmented patient anatomy may also be deployed. This component accounts for the potentially tortuous patient vessel anatomy.

### BRIEF DESCRIPTION OF THE DRAWINGS

FIGS. 1A and 1B are diagrams showing an infrarenal aortic aneurysm (FIG. 1A) and a fenestrated endograft (FIG. 1B).

FIGS. 2A-2D highlight the overall FAM for the selection of an off-the-shelf graft design; input of individual patient anatomy; finding of optimal alignment; and generation of a report to modify a commercially manufactured, readily available thoracic endograft based on patient anatomy.

FIG. 2A is a schematic of the first step in FAM wherein the user selects from a range of commercially available endograft designs. Relevant graft design parameters are indicated for an isodiametric endograft, and a snapshot of a potential user interface used to preview various graft designs in a computational program is shown also.

FIG. 2B is a schematic of the second step in the process, wherein the fenestration positions are determined (for example, by CT scan). Mapping of the graft geometry and fenestrations between the cylindrical (3D) and planar (2D) domain is highlighted. Each fenestration is defined by its proximal graft distance (PGD=z-distance in a cylindrical coordinate system), and arc length (AL=circumferential distance). The angles of the fenestrations φᵢ are used to determine the AL measurements.

FIG. 2C is a schematic of step three; determining an optimal alignment of the fenestrations against the patient's anatomy, and verifying no interference of support structures in the graft with fenestrations.

FIG. 2D is a schematic of step four, in which the results are used to design the graft and produce a 3D visualization of the graft to be implanted.

FIG. 3A is a diagram demonstrating extrapolation of a 2D planar map for the tapered portion of a heterodiametric endograft based on graft design variables h, D₁, and D₂. FIG. 3B demonstrates the piecewise 3D-2D mapping used for the proximal, tapered, and distal portions of the graft respectively. FIG. 3C is a flow diagram highlighting the conditional statements used to determine the mapping transformation required at each axial location of the graft. FIG. 3D illustrates how simultaneous movement of the fenestrations (i.e. rotation or longitudinal motion of the graft) varies based on implementation of the conditional statements in FIG. 3C, wherein fenestrations on the tapered portion of the graft move in a polar coordinate system and fenestrations on the uniform diameter portion of the graft move in a cartesian coordinate system.

FIG. 4 is a diagram of a sample device 100 that takes the selected alignment as an input, and automatically marks or cauterizes the fenestrations in the correct location on the graft. Device 100 in FIG. 4 includes three separate motors 110, 120, and 130. The motors 110 and 120 are operably connected to lead screws 112 and 114. A grip 140 is operably connected to the leveling rod 114. A marker tool 150 is secured on the grip 140. The motor 130 is operably connected to a stand 170 containing a rotating rod 176 secured on two opposing brackets 172 and 174. A mandrel 180 and a graft 200 are positioned over the rotating rod 176. The marker tool 150 may be any marker to mark a location of a fenestration, or a cauterizing tool to generate a fenestrated graft. Thick arrows indicate direction of motion.

FIG. 5 is a flow diagram of the 3D visualization method, including the following elements: the planning method which constitutes measurement of the fenestrations and an optimized search, and the visualization method, which is composed of the mesh generation, mesh parameterization, and texture mapping methods.

FIG. 6A summarizes the mesh generation method, whereby the vessel centerline data is used to determine a spline of best fit appropriate for lofting of the graft 3D mesh. FIG. 6B illustrates the mesh parameterization process which utilizes the discrete Laplacian matrix to generate a 2D representation of an arbitrary 3D graft mesh. FIG. 6C indicates the barycentric coordinate system used for an arbitrary 3D graft mesh element for implementation of texture mapping across its neighboring faces. The point P is the 3D cartesian space (x, y, z) expressed in terms of the barycentric coordinates λ1, λ2 and λ3.

FIG. 7 is a diagram of a sample device 300 wherein fenestrations are created on the medical implant 310 in-situ (intraoperatively inside the body) using a minimally invasive robot that uses integrated imaging and a laser/cautery tool. The minimally invasive robot may use one of several standard medical imaging modalities 320 (e.g. intra-cardiac echiography, intravascular ultrasound, optical coherence tomography) to image the graft support structures 330. The FAM algorithm determines the optimal location for the fenestrations on the medical implant. The implant is modified by pointing a cautery tool/laser beam 340 at the appropriate location, while continuous feedback control is offered by the imaging modality.

### DETAILED DESCRIPTION OF THE INVENTION

Aortic aneurysms are the 13^{th} leading cause of death in the United States. While aneurysms can occur along the entire length of the aorta, the infrarenal location is the most common. Targeted ultrasound screening has been found to be an effective and economical means of preventing aortic aneurysm rupture. The indication for repair includes either symptomatic aneurysms or aneurysms with a diameter greater than 5.4 cm.

The standard definition for an infrarenal AAA is a transverse aortic diameter ≥3.0 cm. Other studies have used a definition of 1.5 to 2.0 times the normal adjacent aortic diameter. Risk factors associated with increased infrarenal aortic diameter include male gender, age, smoking, hypertension, and family history. The approximate incidence of infrarenal AAAs in patients over the age of 65 is 1.7% in women and 5% in men.

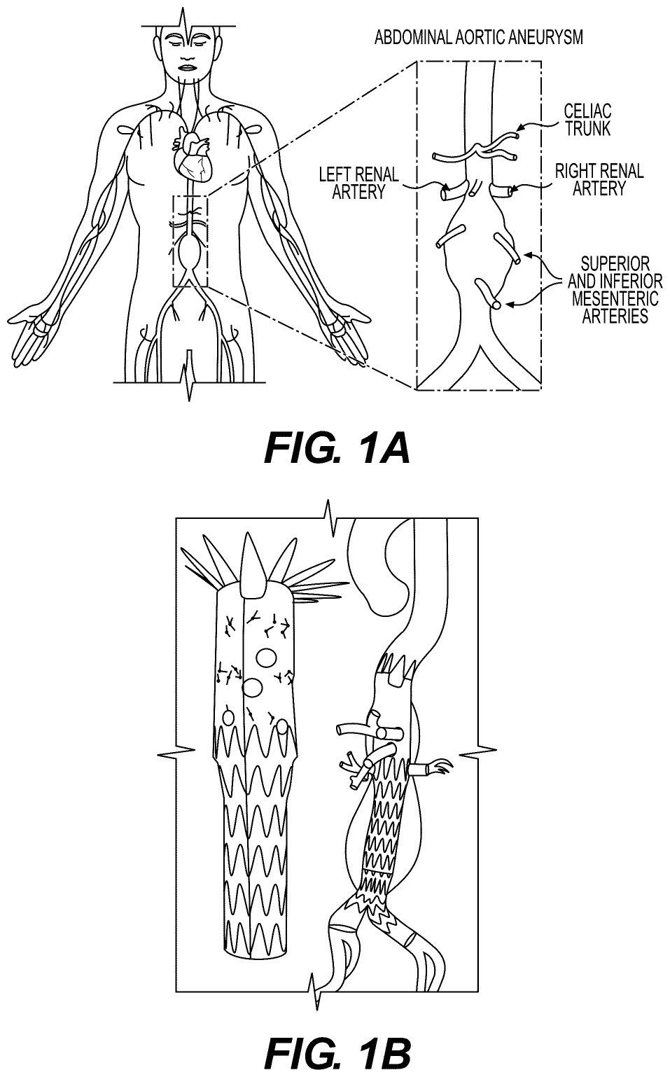

Treatment options for the repair of infrarenal aortic aneurysms are open surgical repair (OSR) and endovascular aneurysm repair (EVAR) where stents are inserted. Currently, EVAR is the primary treatment method for the repair of infrarenal aortic aneurysms due to improved short-term morbidity and mortality outcomes. However, aneurysms that involve the visceral vessels cannot be repaired using a standard commercial infrarenal endograft and instead, fenestrated endografts are needed to maintain blood flow to the visceral vessels. FIGS. 1A and 1B are diagrams showing an infrarenal aortic aneurysm (FIG. 1A) and a fenestrated endograft (FIG. 1B).

The Problem

Standard endovascular grafts are unsuitable for treating juxtarenal AAAs or TAAA aortic aneurysms due to presence of visceral vessels. If a patient presents with a juxtarenal AAA or TAAA aneurysm, physicians can utilize a fenestrated aortic endograft by manually creating holes in standard thoracic endografts grafts to maintain blood flow to the visceral arteries.

This method is guided by examination of individual patient preoperative computed tomography (CT) scans, but is tedious, time-consuming, and involves trial and error on the physician's part.

Biomedical engineering companies do provide a service for creating fenestrated endografts where the contractor will measure and manufacture a hand-made, fenestrated graft. The lead time for this process can be in the order of a few weeks, which is not a feasible time frame in the case of a patient that presents with an urgent condition requiring immediate medical intervention. This special service is only offered to certain institutions. The service is also somewhat detached from the planning procedure, so it is difficult for the physician to preview the final product before it is manufactured and shipped.

### I. Definitions

As used herein, the term “fenestration” refers to an anatomical opening in a vessel, or an opening, a hole, or a cut out on an implant.

As used herein, the term “fenestration alignment” refers to positioning or a position on an implant that corresponds to a position of a fenestration specific to patient's anatomy at the site of implantation.

As used herein, the term “collide” or “collision” refers to physical contact or overlap of the fenestration location with a structural component of an implant, or a digital contact or overlap of pixels designating a fenestration location with pixels designating a structural component of an implant. Typically, the structural component is any one of struts, wires, and stitches.

As used herein, the term “operably connected” refers to a connection between one part of a device to another through function rather than direct connection. The connection may be through other device parts, and/or through wireless communication.

As used herein, the term “patient-specific” describes an object with characteristics specific to, or a process that yields an object with characteristics specific to, a patient's anatomy at the site of implantation of the object.

Recitation of ranges of values herein are merely intended to serve as a shorthand method of referring individually to each separate value falling within the range, unless otherwise indicated herein, and each separate value is incorporated into the specification as if it were individually recited herein.

### II. Methods for Automated Fenestration Alignment

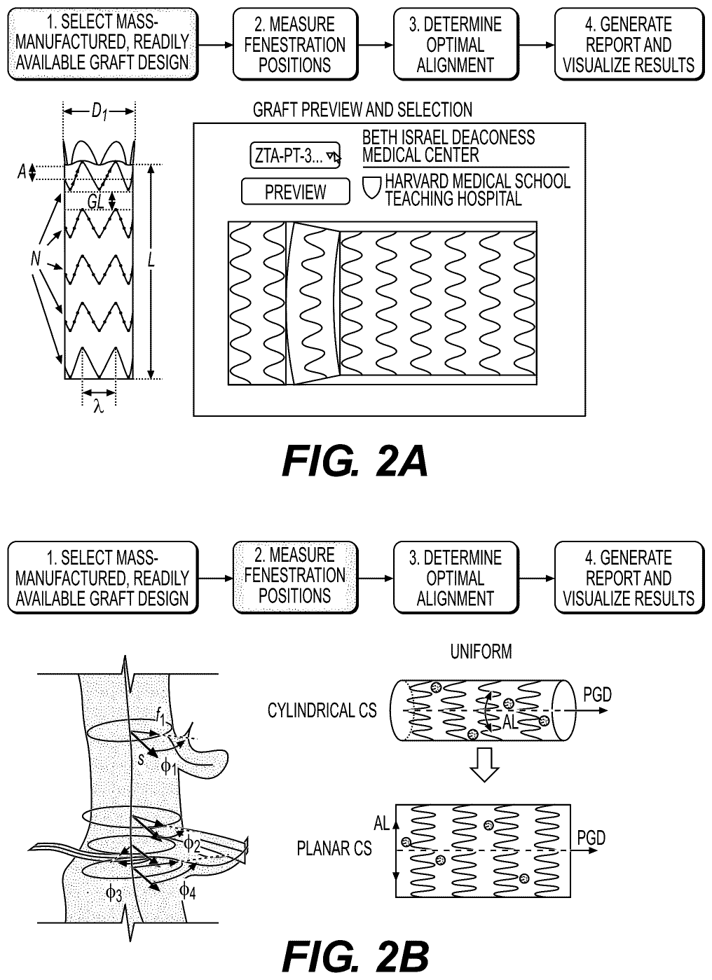

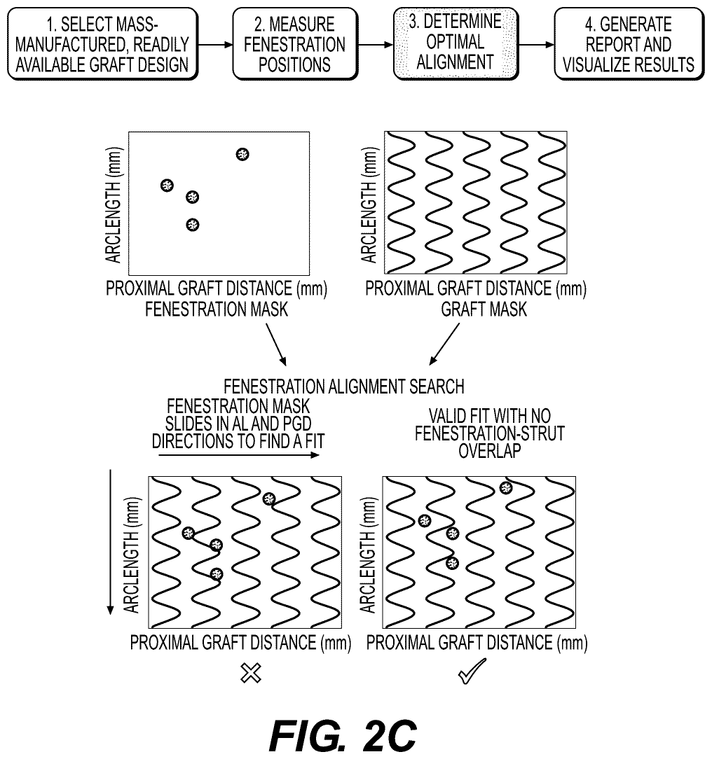

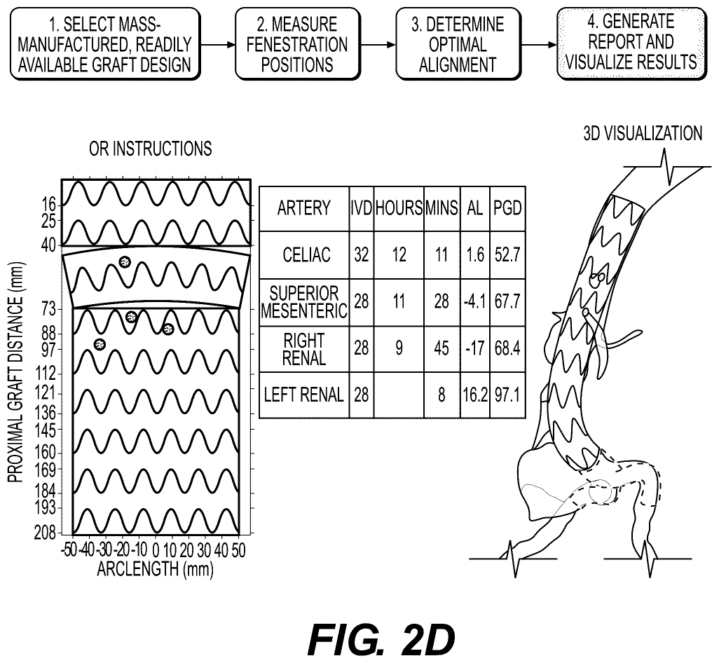

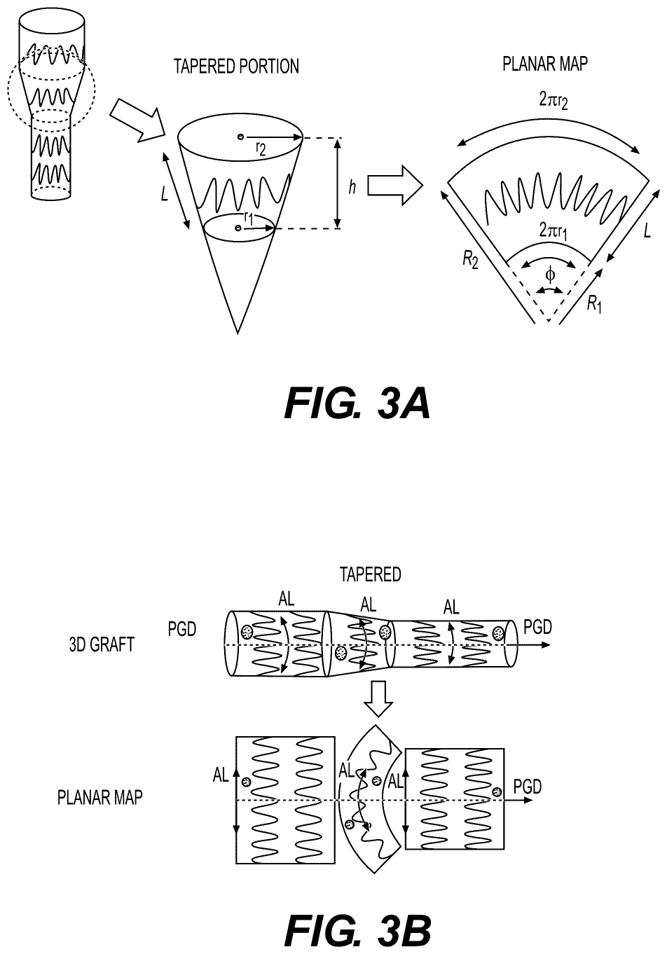

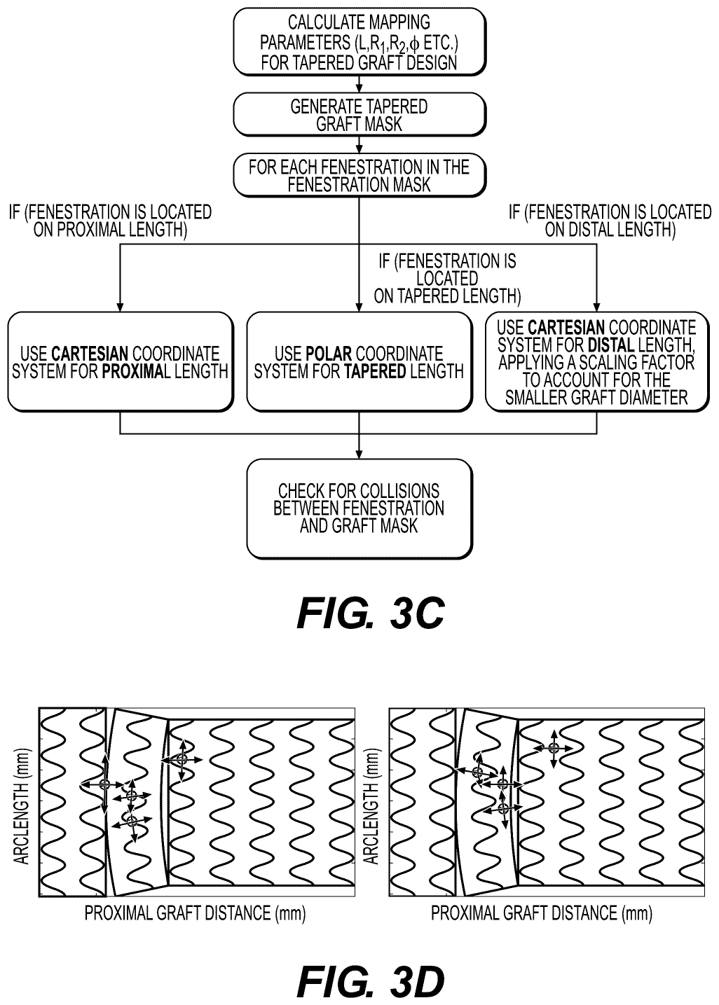

CT data is used to determine the proximal and circumferential positions of each fenestration. Currently, the physician performs hand calculations to determine an alignment using an off-the-shelf graft design; and the graft is inserted into the patient. This contrasts with the Fenestrated Alignment Method (FAM) presented here, in which an off-the-shelf graft is identified (FIG. 2A), information on individual patient anatomy is inputted into the program (FIG. 2B); the optimal alignment determined and avoidance of fenestrations with structural features (FIG. 2C); and a report generated showing a configuration that does not intersect with the stent struts (FIG. 2D), so that the off-the-shelf graft can be accurately modified to match fenestration locations in the graft while reinforcing the aneurysm.

The methods for fenestration alignment in implants, such as endovascular grafts, typically include an automated step of generating fenestration coordinates on an implant. The methods may include a further automated step of marking and making the fenestration coordinates on the implant. Methods may also include a further automated step of modifying the implants at the fenestration coordinates.

A. Selecting Non-Fenestrated Graft Template

The first step of the FAM is to requires selecting a mass-manufactured, non-fenestrated endograft. FAM is parametric, and therefore, variables will be used to represent the key design parameters of the graft rather than specific values (see FIG. 2A).

B. Generating Fenestration Coordinates

FAM typically uses a graft and vessel reference system. Here, the reference system uses two variables representing the location of each fenestration, Arclength (AL) and Proximal Graft Distance (PGD). The values for AL and PGD are measured in mm, and each fenestration's coordinates are defined by its unique AL and PGD combination.

Typically, the values for AL define a location along the circumference of an implant in either direction from a central longitudinal axis (−/+ representing anticlockwise and clockwise directions respectively). The second step of FAM involves measurement of the fenestration positions relative to the aortic anatomy so they may be used in the optimized search method of step three.

Automated Measurement of Fenestration Positions

During conventional fenestration planning, AL measurements are measured relative to the sagittal plane direction [Zhu et al., Fenestrated Thoracic Endovascular Aortic Repair Using Physician Modified Stent Grafts For Acute Type B Aortic Dissection With Unfavorable Landing Zone, Eur J Vasc Endovasc Surg., 55(2), 170-176, doi: 10.1016/j.ejvs.2017.11.0121. A surgeon usually takes these measurements by scrolling through axial CT scan slices and manually inspecting the angle of each fenestration relative to the sagittal plane. An automated method for measurement of fenestration positions is based on the segmented CT scan anatomy, to be used in place of manual inspection of the raw CT data. This method is implemented within the overall FAM workflow.

Surgeons use an “o'clock” reference system in defining the fenestration positions (see FIG. 2B), i.e., the angle left or right of a 12 o'clock datum at a cross section of the vessel. The sagittal basis vector, s, can be projected onto the top and bottom graft profile plane to act as a reference basis vector. The fenestration vector, f, is the vector that joins the location of the fenestration to its closest point on the aortic centerline. The fenestration angle (or angle relative to the 12 o'clock datum) can be is calculated from a rearranged form of the cross-product formula:

$${φ}_{i} = {sin}^{- 1} ( \frac{f \times s}{\vert f \vert \vert s \vert} ) \qquad ( 1 )$$

This angle may be scaled by the vessel diameter to determine the corresponding AL measurement. The axial location of each fenestration along the graft is the PGD measurement.

C. 2D Optimal Search Method

FIG. 2C is a schematic illustrating the third step of FAM; determining an optimal alignment of the fenestrations against the patient's anatomy and verifying no interference of support structures in the graft with fenestrations. In FAM, an automated process is utilized to calculate the location of one or more fenestrations on a two-dimensional (2D) rendering of the implant while avoiding collision with the structural component(s) of the implant.

The structural components of an implant typically include struts, wires, seams, or stitches, that hold the shape of the implant. The method utilizes the information about the location of the structural components and the inputted predicted position of a fenestration to provide the best-suited location for the fenestration that avoids “collision” with any of the structural components of the implant. Collision is any of overlap, touching, or crossing of the structural components. For example, sinusoidal metallic struts support endoluminal grafts. The fenestration holes cannot “collide” with one of the struts as it would not be possible to cauterize the fenestration holes through a metallic strut. Moreover, removal of a portion of the strut would compromise the structural integrity of the graft, which may cause overdilation under excessive loading. One of the goals of the method is to make stents which do not have “collisions” between the fenestrations with the struts.

A mask of all fenestrations can be generated by setting all pixels within a radius of R of the fenestration centers to a value of ‘1’. The distance between fenestrations in the patient anatomy remains fixed. To detect a “collision”, FAM checks if any pixel value sums to ‘2’ after adding the stent strut mask to the fenestration mask. The challenge lies in determining an optimal longitudinal and rotational orientation for a particular graft design (which would correspond to movement of the patient anatomy in 2 dimensions). The fenestration mask is successively convolved with the graft mask in the planar AL and PGD directions to determine a valid fit. This concept is highlighted in FIG. 2C.

Considerations for Tapered Grafts

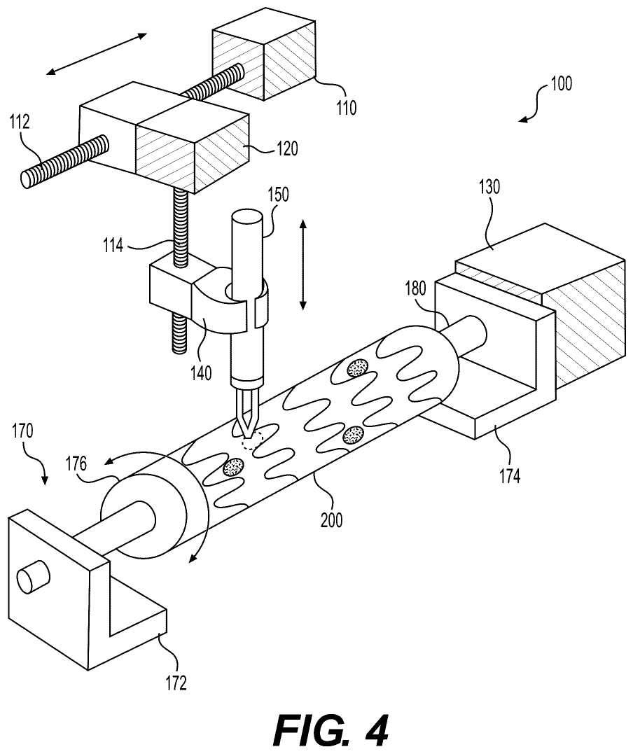

Additional mapping transformations are necessary when considering convolution of the fenestrations on a tapered endograft (as opposed to a standard uniform diameter graft). FIG. 3B is a flow diagram demonstrating the conditional statements used to determine whether cartesian or polar movement are applied to each fenestration. FIG. 3C demonstrates the corresponding fenestration movement on the proximal, tapered, and distal portions respectively.

The tapered portion of the graft can be thought of as a truncated cone or frustum. Its flattened 2D equivalent is a radially truncated circle sector (see FIG. 3A). FIG. 3A is a diagram demonstrating extrapolation of a 2D parameterization of the tapered portion of a heterodiametric graft based on the geometric parameters, D₁, D₂, and h.

Using knowledge of the tapered graft design parameters D₁, D₂, and h, the variables in FIG. 3A can be calculated from simple trigonometry:

$$L = \sqrt{{( {r}_{2} - {r}_{1} )}^{2} + {h}^{2}} \qquad ( 2 )$$

$${R}_{1} = \frac{L {r}_{1}}{{r}_{2} - {r}_{1}} \qquad ( 3 )$$

$${R}_{2} = L + {R}_{1} \qquad ( 4 )$$

$$φ = 360 * \frac{{r}_{2}}{{R}_{2}} \qquad ( 5 )$$

where r₁ and r₂ are the large and small diameters of the graft respectively. Given R₁, R₂ and opening angle c, a binary image mask can be generated. A planar representation of the tapered cosine wave can be generated using a product of cosines (i.e., a sine wave on a pitch circle).

For uniform grafts, a fixed distance remains between the fenestrations on a 2D template. In the case of tapered grafts, fenestrations do not move in parallel directions as the graft is moved rotationally and axially. Conditional statements are necessary to vary the mapping transformation depending on the PGD of each fenestration (i.e., the larger diameter (1), tapered (2), or smaller diameter (3)) (see FIG. 3B).

For a given graft rotation, fenestrations aligned on the smaller diameter of the graft will move a shorter distance than those on the larger diameter. Therefore, a scaling factor k, defined by

$$\frac{{D}_{2}}{{D}_{1}} ,$$

should be used. The Cartesian PGD-AL axes on the uniform diameter coordinate system become polar r, θ axes on the tapered portion. The tapered fenestrations move in an arc on the planar map during rotation of the graft (see FIG. 3C).

D. 3D Visualization of the Final Fenestrated Graft Design

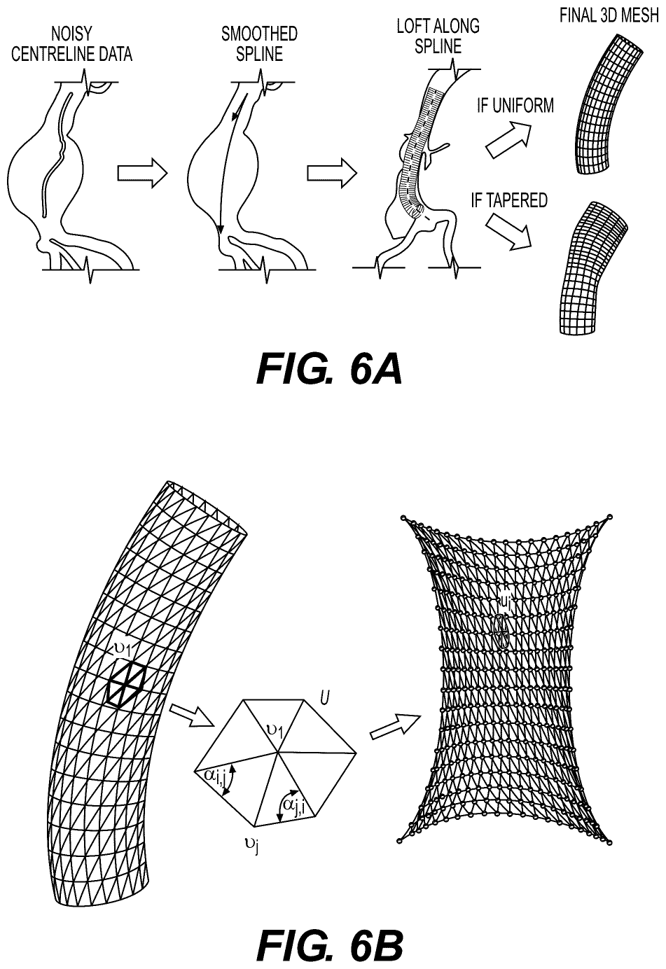

3D visualization of the final fenestrated graft design allows the surgeon to compare the graft design against the initial segmented anatomy, offering an intuitive understanding of the relationship between 2D templates and 3D aortic anatomy, as well as an ideal graft configuration to aim for intra-operatively. FIG. 5 is a flow diagram of the entire FAM workflow, including the visualization method. The FAM method can be applied to all non-manifold graft geometries, facilitating flexibility to introduce more diverse graft designs in the future such as bifurcations and graft branching.

Graft Mesh Generation

The first component of the visualization method is to generate a 3D cylindrical graft mesh suitable for texture mapping. Graft normals are first calculated based on CT vessel centerline data. A circular profile is then swept along a spline of best fit to obtain the final cylindrical graft mesh.

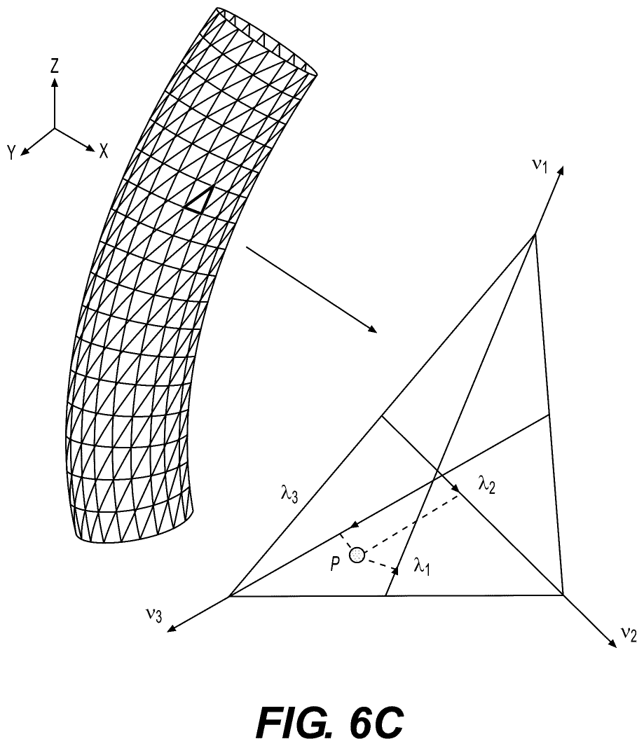

To determine the direction of the normals at the top and bottom of the graft, Singular Value Decomposition (SVD) is applied to the first 10% and last 10% of points along the aortic centerline to determine a 3D line of best fit. Graft endpoint normals for a noisy aortic centerline generated using the SVD approach are illustrated in FIG. 6A.

The program takes the patient's graft diameter selection as an input, and outputs a base and top graft profile appropriate for sweeping along the best fit cubic spline. A 3D mesh of the graft geometry can be generated using a standard lofting function.

2D Mesh Parameterization

To render an aligned planar configuration of the graft on the surface of this mesh (see FIG. 2C), a bijective mapping function (or mesh parameterization) is required between the PGD-AL space and the deformed graft surface in 3D.

A discrete Laplacian matrix is used which describes the degree (connectivity), and adjacency (distance) between vertices on a discrete grid [Strang, Computational Science and Engineering, Wellesley-Cambridge Press, 20071:

L=D−W (6)
where L is the Laplacian matrix, D is the degree matrix, and W is the adjacency matrix. D is a diagonal matrix, which is m×m m×min in size, where m is the number of vertices in the domain. This matrix represents the number of nearby vertices to which any given vertex is connected. W is also an m×m matrix, where a non-zero off-diagonal term (i,j) represents a connection between the vertices and via an edge.

Equation 6 retains information about connectivity and spacing under a 3D to 2D mapping transformation, though the operation is not necessarily angle-preserving (i.e., conformal). To refine the Laplacian matrix for this application, the canonical cotangent-weight Laplacian matrix is implemented. Cotangent weights preserve angles (and consequently areas) more accurately than the standard Laplacian matrix during reconstruction, providing a smoother overall appearance of the mesh. First proposed by Pinkall and Polthier [Pinkall and Polthier, Computing discrete minimal surfaces and their conjugates, Experimental Math, 2(1), 15-36, 1993], cotangent weights provide an indication of the weighted sum of all edge lengths and angles adjacent to the vertex. The weights are calculated by:

$${L}_{i j} = \{ \begin{matrix}{w}_{i j} = \frac{1}{2} ( cot {α}_{i j} + cot {β}_{i j} ) & i f ( i , j ) is an edge \\ {w}_{s u m} = - \sum {w}_{k} & for i = j \\ {w}_{i j} = 0 & otherwise\end{matrix} \qquad ( 7 )$$

FIG. 6B shows the relevant geometry for calculating cotangent weights at given vertex i. In the formulae above the terms wᵢⱼ correspond to a weighted variant of the adjacency matrix, and the terms wsum represent a weighted degree matrix. Together these terms combine to yield the discrete weighted Laplacian in a similar form to Equation 6.

FIG. 6B illustrates a sample 3D graft mesh, relevant geometry at a given vertex i, and corresponding planar geometry generated using the mesh parameterization algorithm as described herein. These figures show the generation of a bijective 3D to 2D vertex mapping for a uniform graft.

Texture Mapping Method

Given that the mesh generated using the discrete Laplacian transformation is tetrahedral, barycentric coordinates are used to discretize and interpolate the planar fenestration map across the 3D surface of the graft.

Under a barycentric coordinate system, the location of a point within a triangle in 3D space is defined as a sum of weights located at its vertices. FIG. 6C shows the barycentric coordinate system for a given triangular mesh face. The point P in the 3D cartesian space(x,y,z) is expressed in terms of the barycentric coordinates (λ₁,λ₂,λ₃).

To discretely interpolate the planar fenestration map across the graft's surface, one can write the Cartesian coordinates of a point r on the graft's surface can be written as a function of both the barycentric coordinates and the local triangular vertices, as follows:

x=λ₁x₁+λ₂x₂+λ₃x₃ (8)

y=λ₁y₁+λ₂y₂+λ₃y₃ (9)
where x and y define a point bounded within the triangular vertices (x₁,y₁), (x₂,y₂), and (x₃,y₃), and λ₁, λ₂, and λ₃ are the barycentric coordinates that sum to λ₁+λ₂+λ₃=1. It is assumed that the mesh is differentially flat and approximately 2 dimensional at local points on the graft.

An inverse mapping of the above equations is required; that is, a transformation that converts (AL,PGD) coordinates within the planar map to barycentric coordinates suitable for rendering in the 3D graft space. To obtain such a transformation, the identity λ₁+λ₂+λ₃=1 is rearranged, and substituted into the Equations 3 and 4 above,

x−x₃=λ₁(x₁−x₃)+λ₂(x₂−x₃)

y−y₃=λ₁(y₁−y₃)+λ₂(y₂−y₃)
or in matrix form.

$$( r - {r}_{3} ) = T λ$$

$$where$$

$$T = [ \begin{matrix}{x}_{1} - {x}_{3} & {x}_{2} - {x}_{3} \\ {y}_{1} - {y}_{3} & {y}_{2} - {y}_{3}\end{matrix} ]$$

Finally, to obtain the desired mapping, one inverts the matrix T

λ=T⁻¹(r−r₃) (10)
where, explicitly, the components of T⁻¹ can be calculated by,

$${λ}_{1} = \frac{( {y}_{2} - {y}_{3} ) ( x - {x}_{3} ) - ( {x}_{3} - {x}_{2} ) ( y - {y}_{3} )}{det ( T )} \qquad ( 11 )$$

$${λ}_{2} = \frac{( {y}_{3} - {y}_{1} ) ( x - {x}_{3} ) - ( {x}_{1} - {x}_{3} ) ( y - {y}_{3} )}{det ( T )} \qquad ( 12 )$$

To use the equations above in practice, each triangular mesh face is first discretized into barycentric coordinates using Equations 11 and 12. For each face on the deformed 2D mesh, its corresponding 3D vertices are extracted, and a 1-1 mapping is assigned between pixels (i.e., 2D discrete elements, on the planar map and their corresponding barycentric coordinates in 3D space. Finally, the planar map is interpolated across the graft's surface, according to each pixel's mapping via the barycentric coordinate system.

E. Displaying the Results

The display of results may include displaying a two-dimensional rendering of the implant with the fenestration alignment. The display of results may include displaying a three-dimensional rendering of the implant with the fenestration alignment. The display of results may also include displaying numerical coordinates for the fenestration alignment. The coordinates typically include the values for AL and PGD. An example of such a display is shown in FIG. 2D. The display allows an operator to evaluate the suitability of the coordinates and/or the rendered implant models with the anatomy of the patient at the location of the implantation.

The methods are suitable for patient-specific fenestration alignment on flat, two-dimensional implants, on linear isodiametric implants as well as on implants of irregular shape, such as tapered (heterodiametric) implants.

### III. Devices for Automated Fenestration Alignment

The automated methods of fenestration alignment typically produce coordinates for fenestrations of a graft in a two-dimensional form. These coordinates can be used to render a model 3-dimensional graft with the fenestrations. These coordinates may also be used to mark commercially available implants for accurate positioning of fenestrations specific to patient's anatomy. These coordinates may also be used to modify commercially available implants and form fenestrations at locations specific to patient's anatomy.

Typically, the implant is marked or modified using an automated device. The device may include a processor that receives the coordinates. The processor then guides one or more motors operably connected to a marker tool to mark or modify the implant at the designated coordinates.

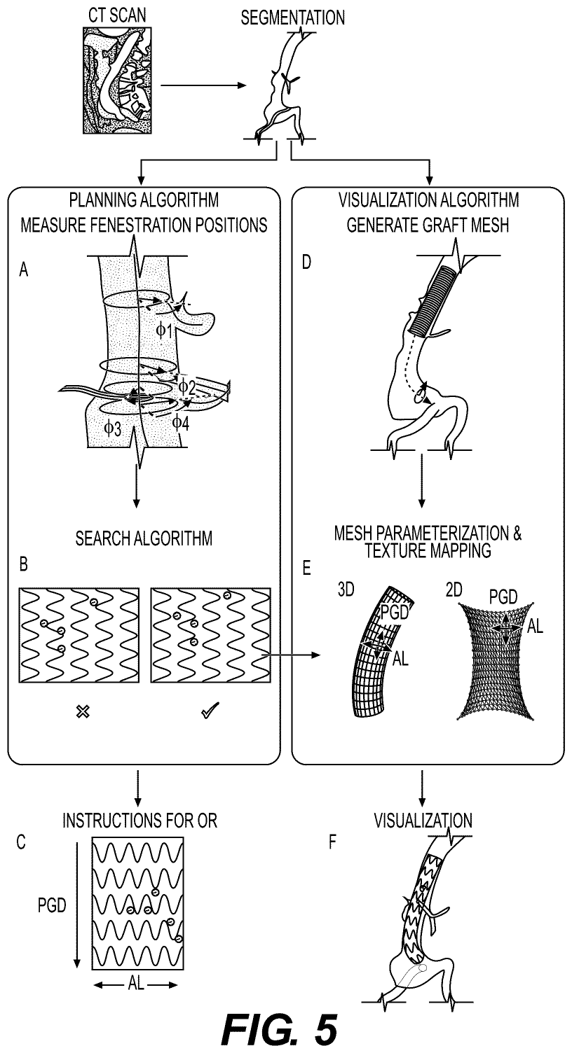

An exemplary device is shown in FIG. 4. The diagram in FIG. 4 shows a sample device 100 that takes the selected alignment as an input, and automatically creates a template, either by directly marking the graft, or laser cutting a plastic sleeve that would then be wrapped around the graft. Device 100 in FIG. 4 includes 3 separate motors 110, 120, and 130. The motors 110 and 120 are operably connected to lead screws 112 and 114. A grip 140 is also operably connected to the leveling rod 114. A marker tool 150 is secured on the grip 140. The motor 130 is operably connected to a stand 170 containing a rotating rod 176 secured on two opposing brackets 172 and 174. A mandrel 180 and a graft 200 are positioned over the rotating rod 176. The marker tool 150 may be any marker to mark a location of a fenestration, or a cauterizing tool to generate a fenestrated graft. Double-headed arrows indicate direction of motion.

Devices with similar parts and arrangements may be used for automated marking of tissues and grafts. Those of skill in the art would envision modifications to the sample device 100 or other devices that receive an input of coordinates and are structures to mark those coordinates on a surface, such as a graft, tissue, or implant.

Several methods can be used to modify the marked endograft (e.g. laser, cautery tool, cutting balloon angioplasty), which could be substituted for the marker tool 150. The fenestration will then be reinforced with a PTFE cuff around the circumference of the graft to prevent potential tearing of the endograft material caused by stenting of the fenestration branches.

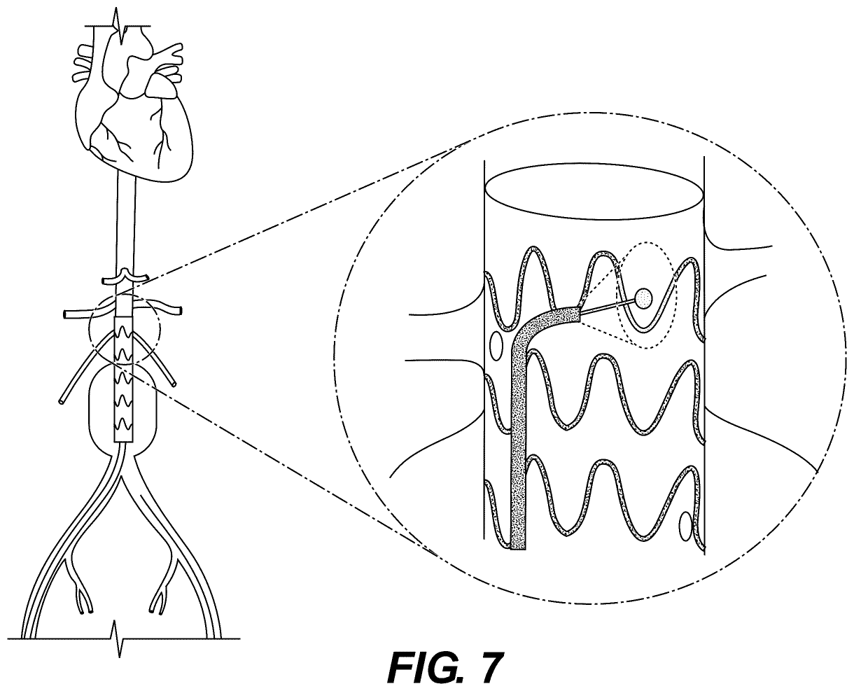

In another embodiment of FAM, the fenestrations are created intraoperatively using a minimally invasive robotic device. FIG. 7 is a diagram of a sample device 300 wherein fenestrations are created on the medical implant 310 in-situ (intraoperatively inside the body) using a minimally invasive robot that uses integrated imaging and a laser/cautery tool. The minimally invasive robot may use one of several standard medical imaging modalities 320 (e.g. intra-cardiac echocardiography, intravascular ultrasound, optical coherence tomography) to image the graft support structures 330. The FAM algorithm determines the optimal location for the fenestrations on the medical implant. The implant is modified by pointing a cutting tool/dilator/cautery tool/laser beam 340 at the appropriate location, as defined by the planning tool, while continuous feedback control is offered by the imaging modality.

### IV. Using the Automated Method and Devices

The devices and methods may be used in a clinical setting by an operator, such as a surgeon, to obtain a patient-specific fenestrated implant. The operator may examine the coordinates for the fenestrations on a two-dimensional or a three-dimensional rendering of the implant and identify if further modifications are needed to the coordinates.

The operator may then manually modify the implant using the coordinates obtained through the automated method. The operator may use one of the exemplary devices to mark or modify the implant at the coordinates obtained through the automated method.

The methods and devices resolve the lack of ready-to-use commercially available patient-specific implants.

The present invention will be further understood by reference to the following non-limiting examples.

### EXAMPLES

### Example 1. Manually Modified Mass-Manufactured, Readily Available Endografts Based on Individual Patient Aortic Anatomy (Prior Art)

Workflow

Surgeons typically use the following workflow to modify mass-manufactured endografts:
1. Using CT imaging, determine the longitudinal circumferential locations of each fenestration that might be covered by an endovascular graft via the implantation in the abdominal aorta.
2. Use trial and error to manually calculate a longitudinal position and orientation of the graft that maintains the patency of the fenestrations, without needing to cut or remove the stent structure. The stent structure is critical for ensuring the graft does not see excessive dilation, which in turn can cause large deformation in the surrounding aortic tissue.
3. Use a cautery tool to manually burn the location of each fenestration in an infrarenal endovascular graft, based on the results of the alignment hand calculations.

Step 2 in particular is the most tedious and time-consuming, and often prone to error unless extreme care is taken with each calculation.

### Example 2: Graft-Fenestration Alignment Program

A computer program was developed to improve the accuracy and efficiency workflow issues highlighted in Example 1. The program is referred to as “FENFIT™”. This method automates the alignment hand calculations that are typically conducted by the physician. The purpose of FENFIT™ is to provide the following:

Provide the physician with an intuitive, visual user interface for modifying endovascular grafts based on underlying patient-specific anatomy.

Reduce the overall workflow time to perform the alignment process. as compared to a manual physician approach.

Allow the physician to modify any graft from a flexible graft design repository, facilitating modification of both tapered and uniform diameter grafts.

Provide a summary of the primary results in a standardized format, so that design selections can be referenced and communicated amongst physicians.

Render the modified graft selection on screen in 3D for the physician, to give them confidence the method has completed its task accurately.

Automated Alignment on Linear Grafts

Operating software, such as MATLAB, can be used to implement FAM, to expedite search of a viable design parameter space for the graft, while accounting for the constraints imposed by the patient's underlying anatomy and endograft structural constraints. Computationally, it is only feasible to divide the graft into discrete parts, such that the user can only select from a finite number of fenestration placement configurations. MATLAB's image processing functionality may be utilized in PENFIT™ to easily manipulate these discrete components as pixels in a 2D spatial grid.

After selecting an optimal design, the program may render the suprarenal graft on screen for the physician to facilitate improved visualization of the graft for the physician.

The method may also include using the operating software to instruct a device with the graft positioned on a mandrel to produce the fenestrations unique to the patient.

The method automatically designs suprarenal grafts, custom fitted to each patient's unique aortic anatomy based on CT scan data.

The method designs and deploys a way for automated alignment of fenestrations along suprarenal endovascular grafts.

Graft Design Programming

Any graft can be inputted to FENFIT™, provided it is given the appropriate information about its design parameters.

The footprint of this flattened cylindrical graft is a rectangle of size (πD₁×L). Initially a binary mask template is generated, where ‘1’ represents a pixel containing a strut, and ‘0’ represents a free area of the graft.

Vectors PGD and AL represent the fenestration locations and R represent their radii, and stent struts are generated using parametric cosine functions.

A 2D search algorithm is used. A mask of all fenestrations can be generated by setting all pixels within a radius of R of the fenestration centers to a value of ‘1’. The distance between fenestrations in the patient anatomy remains fixed. To detect a “collision”, FENFIT™ checks if any pixel value sums to ‘2’ after adding the stent strut mask to the fenestration mask. The fenestration mask is successively convolved with the graft mask using a for loop in both the planar AL and PGD directions to determine a valid fit. (see FIG. 2C).

User Interface Design

Selecting Graft Design and Inputting Patient Anatomy

When the user first enters the program, they are prompted to select a design from an endograft repository. The user can preview any graft on screen.

On the second panel, the user has two options—automatic entry or manual entry. Under manual entry, the surgeon enters the aortic diameters, PGD, AL, and radius values for each of the fenestrations. The user can then modify the number of fenestrations to be included in the alignment and provide a label identifier for each.

Drag/Drop Functionality and Report Generation

Once the user has saved the patient anatomy configuration, the alignment panel is presented. The user can drag and drop the fenestrations such that they do not intersect with the adjacent stent struts. As the fenestrations are dragged and dropped across the screen, label identifiers are presented adjacent to each, which include information of their current PGD and AL distances.

Report Generation

When the user is content with their alignment selection, the final step of the program is to render the fitted graft on screen and generate a report. The 3D rendering component was described in the detailed description of the invention. FENFIT™ fills a report template automatically based on the user's final selected alignment. This template can be modified for other hospitals/physicians, based on their preferred report formatting. An example of a report output from FENFIT™ is shown in FIG. 4. FIG. 4 shows an auto-compiled report showing all calculated locations the physician needs, as well as a printout of the selected alignment.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. An automated method for fenestration alignment on a medical implant, the method comprising
obtaining fenestration location and radius from patient's anatomy,
aligning the fenestration location and radius on the medical implant such that the aligning does not collide with a structural component of the implant, and
optionally, marking the fenestration location on the medical implant.

2. The method of claim 1, wherein obtaining the fenestration location and radius from patient's anatomy comprises obtaining Proximal Graft Distance (PGD) and Arclength (AL) for each fenestration.

3. The method of claim 2, wherein the patient's anatomy is represented with a non-invasive imaging of computerized tomography (CT), magnetic resonance imaging (MRI), or radiography.

4. The method of claim 2, wherein obtaining fenestration location and radius comprising obtaining values for Proximal Graft Distance (PGD) between about 0.1 mm and about 500 mm.

5. The method of claim 2, wherein obtaining fenestration location and radius comprising obtaining values for Arclength (AL) between about −100 mm and +100 mm.

6. The method of claim 1, wherein aligning is an automated process utilizing the fenestration location and radius on the medical implant.

7. The method of claim 1, wherein aligning is an automated process permitting digital operations selected from the group consisting of positioning the fenestration location on a two-dimensional reconstruction of the implant, viewing, storing, modifying, printing, sharing, and three-dimensional reconstruction of the medical implant with fenestration location.

8. The method of claim 1, wherein the medical implant is selected from the group consisting of stents, implants, vascular grafts, autologous grafts, allogeneic grafts, xenogeneic grafts, synthetic grafts, and hybrid stent grafts.

9. The method of claim 1, wherein the structural component is selected from the group consisting of struts, wires, seams, and stitches.

10. The method of claim 1 wherein the graft is in situ.

11. The method of claim 7 wherein the resulting three dimensional fenestrated graft is rendered inside the three dimensional segmented patient anatomy.

12. The method of claim 1 comprising aligning the graft aligns with the tortuosity of the vessel into which the graft is to be positioned.

13. A system for marking a fenestration location on a medical implant, the device comprising
a rotating rod for receiving the medical implant,
a marker tool for marking the fenestration location,
one or more motors operably connected with the rotating rod and/or the marker tool,
wherein the one or more motors, when in operation, receive input of the fenestration alignment derived from the method of claim 1.

14. The system of claim 13, wherein the marker tool is operably connected to the one or more motors for positioning the marker tool at the fenestration location.

15. The system of claim 13 comprising a medical implant over the rotating rod.

16. The system of claim 13 comprising a mandrel over the rotating rod, and optionally, a medical implant over the mandrel.

17. The system of claim 13, wherein the medical implant is selected from the group consisting of stents, implants, vascular grafts, autologous grafts, allogeneic grafts, xenogeneic grafts, synthetic grafts, and hybrid stent grafts

18. The system of claim 13, wherein the one or more motors operably connected with the marker tool are connected via lead screws.

19. The system of claim 13, wherein the one or more motors operably connected with the marker tool are connected via lead screws positioned in any three-dimensional position.

20. The system of claim 13, wherein one of the motors is operably connected to the rotating rod and two or more motors are operably connected to the marker too.

21. The system of claim 13, wherein the marker tool is a marker or a cauterizing tool.
